(19) United States
(12) Patent Application Publication
(10) Pub. No.: US 2015/0196340 A1
(43) Pub. Date: Jul. 16, 2015

(54) SURGICAL INSTRUMENT

(71) Applicant: Zbigniew COMBROWSKI, Tuttlingen (DE)

(72) Inventor: Zbigniew Combrowski, Tuttlingen (DE)

(21) Appl. No.: 14/346,572

(22) PCT Filed: Sep. 17, 2012
(86) PCT No.: PCT/EP2012/068249

§ 371 Date: Mar. 21, 2014

(30) Foreign Application Priority Data
Sep. 22, 2011 (DE) 10 2011 053 848.8
Jun. 8, 2012 (DE) 10 2012 104 973.4

(51) Int. Cl. A61B 17/88

(52) U.S. Cl. CPC A61B 17/88; A61B 17/8875

(57) ABSTRACT

A surgical instrument (1, 12) for introducing an implant, comprising a shaft (2), wherein the shaft is suitable for accommodating the implant, in particular a screw (9, 15) having a cannula (4), at the proximal end of the shaft, wherein the shaft (2) has a cannulation, and a handle (3), which is connected to the shaft (2) at a distal end of the shaft (2). The shaft (2) is provided with a slit (6).

### BACKGROUND OF THE INVENTION

The invention relates to a surgical instrument and, more particularly, a shaft for a surgical instrument.

For connecting bone pieces, e.g. in Osteotomies or Traumas, different screw implants are also employed with Snap-Off-Shaft and in conjunction with bone plates.

In order to obtain the desired properties of the bone joints, the screw-length and screw-position of the screw must be matched with the bone-pieces to be joined.

Nowadays, it is done in non-cannulated screws, e.g. by measuring devices, such as bore-depth gauge or e.g. under CT or X-ray, wherein for safety, the screw-length or the bore-depth is always checked with a measuring device found in the OP-Set. Some manufacturers also offer in the set, instruments for manual navigation of the screw-position.

According to the type of the screw, e.g. cannulated screws for determining the screw-length and for secured positioning of the screw, a guide-wire with a defined length is also introduced to the desired depth in the bone pieces. With the help of the length of the guide-wire protruding out of the bone, the bore-depth and thereby also the screw-length can be determined by a length-gauge.

A variety of devices are known from the state of the art, which attempt to simplify the positioning of the implants.

For example, the document DE 20 2005 011 355 U1 discloses a measuring-gauge for determining the depth and the diameter of a bore in a vertebra. For this purpose, a size measuring device is used, which is moveable disposed in a depth-gauge in a. Disadvantageously, in addition to the implant ion tool, the depth-gauge also needs to be stored, inserted and cleaned through the wounds.

In addition, the document EP 0 209 685 A2 discloses a screw-anchor made of an elastic, bio-compatible plastic, which has a thread on its outer surface. The position of the screw-anchor can be monitored on the X-ray screen in conjunction with a metallic screwdriver during their assembly. Disadvantageously, another device is likewise necessary in order to determine the position of the screw.

Furthermore, the document DE 202 03 439 U1 discloses a surgical navigation screwdriver with a fixing-sleeve for screws with external screw-head threads, wherein this has an integrated tracker attachment-sleeve for navigated computer-assisted applications. Therefore, this is a very expensive Operation-Set, which includes many component parts.

The main problem of all the OP-Sets found in the market is the sum of the different devices for conventional OP methods and for the computer-assisted surgery as well as the number of the individual OP steps.

Consequently, the sets are large and are tuned for the individual OPs and need a lot of space for storage. Thus, high costs of preparation and sterilization are involved.

Very often, many OPs are conducted in a day in the fields of small fragments and accident surgery. Thus, the availability of such sets is also a big issue.

In all the mentioned devices, one disadvantage is common that a plethora of different instruments and devices and parts are required for an Operation Set.

In addition, the drilling, countersinking and thread-milling devices in the surgery in cannulated configuration are also manufactured of different medicinally permissible materials. This mostly involves surgical steels. For this purpose, these are additionally hardened or tempered, as required.

Even these instruments for combined configurations, e.g. for drilling, countersinking and/or for thread-cutting are integrated in order to save OP steps.

Depending on the application, the instruments are very long and need to be very flexible, so that they do not break during the application. The blades are hard, mostly tough as well. The object of these hard materials is to drill through all the known structures, e.g. bones, tissues, cartilage or teeth, for example, without heating as far as possible.

Depending on the method of operation, the instruments are inserted under guidance by means of a guide wire, e.g. in a cannulated configuration. For this purpose, the instruments are provided with a through bore, which is designated as cannula.

These cannulae can be manufactured with much difficulty, because these instruments are usually very small and/or very long. The materials are difficult to machine because of their hardness. In addition, the drilling, gun drilling and grinding are very costly manufacturing processes.

A big disadvantage of these cannulated instruments during the operation is also, for example that the material reaches in the long cannula and jams the guide-wire and then rotates therewith. Thus, the guide-wire is inserted deeper than desired, which may lead to complications during the operation. Even cleaning of these cannulae after the operation is very difficult and complex.

Another exemplary embodiment from the state of the art is drill without cannula. In these, preferably two flutes, preferably helical, are made on the outer jacket. These have the object of removing the drilled material out of the bore-hole. Even here, the manufacture very costly, because initially these grooves have to be milled and then ground. Furthermore, small drills are very delicate and can easily break off, especially due to the usual two flutes.

It is an object of the invention to provide a surgical instrument for inserting an implant, which is configured very easily and which facilitates determining the bore-depth, navigation and ease of cleaning. Furthermore, costly manufacturing processes should be avoided.

### SUMMARY OF THE INVENTION

The foregoing object is achieved by providing a surgical instrument as shown and described hereinbelow.

In typical exemplary embodiments, a surgical instrument includes a shaft for inserting an implant.

Preferably, the shaft is suitable to attach an implant at its proximal end, particularly a screw with a cannula. Preferably, the shaft has a piece-by-piece cannula, preferably at one end. This offers the advantage that a guide-wire, a so-called K-Wire can be guided into the shaft.

The cannula is slit open behind it as far as possible, preferably slit-open on one or more sides, or completely. The aim is not to impair the strength of the instrument and to improve the ease of cleaning and sterilization. At the same time, the slit open outer sheath should provide more space, e.g. for graduations or labelling of the instruments. Preferably, at least one part should be cannulated and/or at least one part should be partially slit open. In what ratio are the distances and/or angle, differs depending on the application and instrument. Still more preferably, at least one part should be cannulated and slit open from at least one or more sides, in order to ensure an optimal guidance of, e.g. the guide-wire. In another preferred exemplary embodiment, the slits should be made from several sides, such that these are connected almost in the middle, preferably connected axially and thus a cannula is made, e.g. for a guide-wire. The cannula can also undertake further tasks, e.g. as a channel for inserting a probe or an anchor or other instruments.

The surgical instrument suitably includes a grip which is connected to the shaft at a distal end of the shaft. Preferably, the shaft is firmly connected to the grip.

In alternative exemplary embodiments, the grip is connected to the shaft in a detachable and exchangeable manner. Preferably, the grip includes a coupling member, particularly a plug-in connector, a thread or a latching element, which can be connected to a suitable subject matter at one end of the shaft in a detachable manner.

In typical exemplary embodiments, the shaft has a slit. Preferably, the slit is suitable for connecting the cannula of the shaft with a surface of the shaft. This offers the advantage that the cannula or the slit can be easily manufactured. In addition, it is advantageous that the shaft and the cannula are easy to be cleaned through the open slit.

Another advantage is that a K-Wire or its end guided in the cannula can be observed. Sometimes, it happens that the K-Wire is pushed forward in an undesirable manner while inserting the implant or screwing in the screws. With the slit open cannula, a visual control over the site of the guide-wire or K-Wire is possible for the doctor.

In typical exemplary embodiments, the slit is made on one side. Preferably, the slit is made substantially over the entire length of the cannula.

In alternative exemplary embodiments, the shaft includes a plurality of slits. This offers the advantage that the cannula can be cleaned still better.

In typical exemplary embodiments, a surgical instrument for inserting an implant includes a shaft, wherein the shaft is suitable for accommodating the implant, particularly a screw at its proximal end. Preferably, it involves a thick shaft which is not cannulated and does not include any slit.

The surgical instrument suitably includes a measuring caliper. Preferably, the measuring caliper is suitable for at least approximately determining the bore-depth and/or the implant-length, particularly, the screw-length.

In typical exemplary embodiments, the measuring caliper can be moveably connected to the shaft of the surgical instrument. In typical exemplary embodiments, the measuring caliper includes a catch. Preferably, the catch is clipped on the shaft. Preferably, the measuring caliper is connected to the shaft in a detachable manner. This offers the advantage that the measuring caliper can be combined with different shafts.

In typical exemplary embodiments, the shaft includes graduations. Preferably, the graduations are suitable for determining the implant-length and/or the insertion-depth. Preferably, the graduations are suitable for determining the insertion-depth or the implant-length in cooperation with another element.

In typical exemplary embodiments, the other element is a rear end of the K-Wire or the guide-wire, which is guided in the cannula, and can be seen through the slit.

In typical exemplary embodiments, the other element is the measuring caliper and/or a display of the measuring caliper, which displays the implant-length and/or the insertion-depth on the graduations.

In typical exemplary embodiments the shaft is connected to an implant, particularly a screw, at its proximal end via a breaking off point. Preferably, an implant involves a so-called Snap-off-Implant. Preferably, the implant breaks off after reaching a certain torque at the breaking off point. Preferably, after breakage of the implant at the breaking off point, a carrier is exposed at the proximal end of the shaft. Preferably, the carrier is suitable for gripping into a corresponding mating part on the implant, in order to screw-in the implant, if necessary, still further into the bones. This offers the advantage that the implant cannot be lost during insertion, and the operator can carry out the complete insertion process as well as determination of the screw-length by an instrument.

In typical exemplary embodiments, the shaft has a carrier at its proximal end, suitable to be brought in engagement with implant. In typical exemplary embodiments, the carrier involves a screwdriver blade. Preferably, the carrier is configured as a slit, Torx, Torq, cross, cross-slit, hexagon or Allen. The carrier is suitably exposed at the predetermined breaking point only after the breaking off of the implant.

In typical exemplary embodiments, the shaft has a coupling at its proximal end. Preferably, the coupling is suitable for connecting to an replaceable carrier. Preferably, the carrier involves a screwdriver blade. Preferably, the screwdriver blade has a slit, Torx, Torq, cross, cross-slit, hexagon or Allen.

Preferably, the replaceable carrier is configured in the shape of a Bit or a Nut. Preferably, the implants, particularly, screws are housed sterile-packed or separately for the OP.

In typical exemplary embodiments, the measuring caliper has an end stop or a tip to be applied on the implant-end on the bone, skin or tissues and/or a shaft-end. Preferably, measuring caliper is available in two configurations. Therefore, it is in an extracorporeal and an intracorporeal configuration. Therefore, the extracorporeal configuration of the measuring caliper serves for measurements on thin skin layers from outside and the intracorporeal configuration serves for the direct measurement, preferably though the access on the bones.

Preferably, the measuring caliper includes another tip, which facilitates the doctor during pressing or inserting the tip through the skin and tissues on a bone-side and by pressing the screwdriver blade tip on the other side of the bone, to determine the screw-length and insertion device. The same is also possible instead with the screwdriver, for example, with a drill. If the graduations are made on the outer sheath of the drill, it is advantageously possible to facilitate a navigated drilling or a drilling at a defined depth.

In typical exemplary embodiments, the caliper with the caliper tip is suitable as a navigator for finding the correct drilling or screwing position. Advantageously, the measuring caliper is also suitable as a fixture and counter-support during the screwing-in by hand or by a machine or by a Robot or manipulator. The measuring caliper can also serve as an end stop for a defined maximum bore-depth.

In typical exemplary embodiments, the measuring caliper has an end stop, preferably, a tip for applying an implant-end on the bone, skin or tissues and/or a shaft-end.

Preferably, the measuring caliper is in two configurations, extracorporeal and intracorporeal. The extracorporeal configuration is for the measurement on thin skin layers from outside, the intracorporeal configuration in order to directly measure through the access on the bones.

Preferably, the measuring caliper includes another tip, which facilitates the doctor during pressing or screwing the tip through on/in the skin, bone or/and tissues on one bone-side and by pressing the screwdriver tip on the other side of the bone to determine the screw-length and insertion device.

The same is also possible instead of the screwdriver, for example with a drill. If for example, the graduations are made on the outer sheath of the drill, it is also advantageous here, the navigated drilling, and drilling at a defined depth.

In typical exemplary embodiments, the caliper with the caliper tip is suitable as a navigator for finding the correct drilling or screwing position. Advantageously, the measuring caliper is also suitable as a fixture and counter-support while screwing by hand or by a machine or by a robot or manipulator. The caliper can also serve as end stop, e.g. for a defined maximum bone-depth.

Preferably, the measuring caliper can also be employed in conjunction with surgical instruments, which have a cannulated and slotted shaft. Advantageously, with this, the tissue-thickness which must be pierced, and the depth of screwing-in in the bones can be determined by an instrument.

In typical exemplary embodiments, the surgical instrument in accordance with the invention includes an active or passive tracker. Preferably, the tracker is made on the shaft or grip of the screwdriver. Therefore, preferably the screw-position can be monitored with minimum invasive or computer-assisted OPs. Preferably, the tracker is integrally connected to the surgical instrument. In typical exemplary embodiments, the tracker is configured in order to be detachably fastened on the shaft or grip of the screwdriver.

In typical exemplary embodiments, the shaft has a screwdriver-blade or a carrier in cross-slit, hexagon, Torx or anchor attachment system.

Preferably, all screwdriver-blades are so configured that they form a unit with the shaft and cannot be lost during the OP. The shaft, the carrier and the implant suitably form a rigid body and are non-detachable, in order to facilitate a computer-assisted, precise and secure working.

In typical exemplary embodiments, the shaft includes a double carrier. Preferably, the double carrier is suitable for connecting a non-detachable Snap-Off-Screw or a non-detachable Snap-Off-Implant to the shaft.

In typical exemplary embodiments, the shaft includes a quick-change system, particularly, a bayonet system for connecting an replaceable carrier.

In typical exemplary embodiments, the shaft has a carrier suitable for inserting anchors, Snap-Off-Anchor systems, staples or Snap-Off staples. This offers the advantage that the surgical instrument is suitable for inserting a variety of implants.

In typical exemplary embodiments, the surgical instrument includes a torque limiter. Preferably, the torque limiter is configured as a torque cartridge. In typical exemplary embodiments, the torque limiter can be added to the surgical instrument, as required.

In typical exemplary embodiments, the surgical instrument has a ratchet. This offers the advantage that the surgical instrument can be operated easily. In typical exemplary embodiments, the ratchet includes a torque limiter.

In typical exemplary embodiments, the shaft has a protective sleeve. This offers the advantage that the surgical instrument or the implant can be inserted in a tissue-friendly and tissue-conserving manner. The sleeve is resiliently configured. The sleeve is suitably interchangeable. Preferably, the sleeve is configured conical.

In typical exemplary embodiments, the sleeve is configured slotted. This offers the advantage that, the sleeve can be widened by the screw or by a mechanism, for example during contact with the bones.

In typical exemplary embodiments, the measuring caliper has an integrated holding device for bone fragments, for drill sleeves and/or a template holder as spot-film device for revising the screws. Preferably, the measuring caliper has a holding device for fixing of plates.

In typical exemplary embodiments, the shaft and/or the screwdriver grip has an attachment for a quick-change attachment. This offers the advantage that the surgical instrument can be clamped in an OP-Robot arm. This offers the advantage that the surgical instrument can also be used in robot-assisted operations.

In typical exemplary embodiments, the measuring caliper has a marking. Preferably, the marking is suitable for displaying the measured the implant-length on the graduations of the shaft.

Separately, a protection is claimed for a surgical instrument including a shaft with a cannula and a slit and a measuring caliper. This offers the advantage that the end of a guide-wire can be observed through the slit and monitoring or further comparative measurements can be carried out by the measuring caliper. In particular, the measuring caliper is suitable for determining the tissue-thickness to be pierced-through and for navigation.

Separately, the protection is claimed for a shaft for a surgical instrument with the described features.

Preferably, the implant-magazine is dispensed with in the instruments set because of the better availability and savings by reuse.

In typical exemplary embodiments, standard instruments are dispensed with in the set. Normally, these are available in sufficient quantities at the hospitals.

Preferably, only the OP specific instruments are found in the set, which are housed separately or sterile-packed. This offers the advantage that, the surgical instrument in accordance with the invention can be configured as multi-piece or as one-piece instrument as well.

Since, the shaft and grip are detachably interconnected; this offers the possibility that the shafts in accordance with the invention can be quickly connected to other attachments. This offers the advantage that a use in quick-change or coupling systems is possible by additional components, whereby a quicker change into manual or computer-navigated operation is possible. This offers an increased process safety.

Another advantage is that manual and computer-assisted OP methods can be implemented by the surgical instrument in accordance with the invention. Furthermore, advantageously, the number of the instruments is minimized and the number of the OP steps is reduced by the application of a single instrument. Thereby, the operation is simplified.

Another advantage of the surgical instrument in accordance with the invention is that apart from a measurement function, this also has a comparator function and/or a navigation function. Thereby, the application of additional measurement instruments can be dispensed with.

Preferably, e.g. during the manufacture of non-cannulated drills or stepped bores, spiral grooves on the outer sheath should be dispensed with. These should be only partially slotted, which leads to a suitable manufacture in better quality.

Still more preferably, these drilling instruments which connect the slits made from several sides to at least one position, should still better pierce through, this, e.g. the material of the second blade should also reach into the slit during drilling. This offers the advantage that the material is collected in a slit carefully and can be easily taken out of this, for example, in order to be used anywhere else or to serve as sampling.

Preferably, this technique should be employed in screwing, impacting, drilling, countersinking, milling instruments, instrument shafts or guide sleeves in the surgery, but also e.g. in guide tubes used in the endoscopy.

In principle, the idea of the invention should find application in combinations of different instruments. Thus, for example, combined drilling and countersinking instruments can also be conceived in a cannulated configuration. Here, these instruments should be collapsible in single or multi-piece. All configurations can be generously provided with markings/graduations and/or labelling along the slit on the outer sheath, wherein these combined graduations and/or markings and/or labelling and or colour coding can serve, for example, in determining the screw-length and/or the depth of the guide wire inserted into the bones or drills.

The instruments in accordance with the invention can also be equipped with, e.g. quick-latching systems. These serve, for example, for simple adjustment of the screw-length. All configurations serve individually or also in conjunction with, e.g. a drilling jig, a measuring caliper, a navigator or a combination of these, for improving the positioning, the guidance, as counter-support or end stop. All configurations in accordance with the invention can also be configured as one-piece instruments, which is possible thanks to the cost-effective manufacture. All configurations can also be manufactured as hybrid made of e.g. two materials. Here, it depends on the conditions of price and availability, as laid down by the user. The configurations in accordance with the invention are implemented in such a manner that the shaft and the grip can be firmly interconnected or for example, the grip and the shaft can be connected to a coupling.

The configurations in accordance with the invention is designed with shaft and coupling for Bits or other attachments, in such a manner that Bits can serve e.g. as screw, drill or carrier, e.g. for Anchor.

A greater advantage is the suitable, simple manufacture which is especially very important in single use instruments. However, even in multi-piece instruments, these at least partially slotted configurations are easy, quicker to clean in finishing as well as after the operational defects and to sterilize and therefore are better reproducible and rather correspond to the present hygiene standards for achieving validated cleaning and sterilization processes in the field of medicine technology.

The aim is also not to restrict use these at least partially or completely slotted instruments only for the mentioned instruments, but also in other instruments, which traditionally have cannula.

It is also conceivable employ these at least partially slotted configurations even in commercial tools.

### BRIEF DESCRIPTION OF THE DRAWINGS

The invention will be briefly described in the following with the help of the accompanying drawings, wherein the figures show:

FIG. 1 shows a schematic representation of a side view of a surgical instrument in accordance with the invention, having a slotted shaft and a bone-screw as an implant, which is connected to the shaft via a breaking off point;

FIG. 2 shows a schematic representation of a section with a K-Wire of one of the exemplary embodiment according to FIG. 1;

FIG. 3 shows a schematic representation of a perspective view of a cannulated slotted shaft of the surgical instrument of the exemplary embodiment of the FIGS. 2 and 3;

FIG. 4 shows a schematic representation of a side view of another exemplary embodiment of the surgical instrument with a cannulated slotted shaft and an replaceable carrier with a bone screw in accordance with the invention;

FIG. 5 shows a schematic representation of a section of the exemplary embodiment of the surgical instrument in accordance with the invention, having a cannulated slotted shaft and an replaceable carrier with a K-Wire according to FIG. 4;

FIG. 6 shows a schematic representation of a side view of another exemplary embodiment of a surgical instrument with a measuring caliper in accordance with the invention;

FIG. 7 shows an enlarged section of the measuring caliper and the shaft according to FIG. 6;

FIG. 8 shows another schematic representation of an enlarged section of the measuring caliper and the shaft according to FIG. 6;

FIG. 9 shows a schematic representation of the surgical instrument with a measuring caliper and a bone screw as an implant in accordance with the invention;

FIG. 10 shows an enlarged section of the screw, measuring caliper and the shaft according to FIG. 9;

FIG. 11 shows a schematic representation of another enlarged section of the shaft and the measuring caliper according to FIG. 9;

FIG. 12 shows a schematic representation of a perspective view of the measuring caliper of the FIGS. 6 and 9.

FIG. 13 shows a partially sectioned side view of another exemplary embodiment of an instrument configured in one-piece in accordance with the invention;

FIG. 14 shows a perspective view of the instrument according to FIG. 13;

FIG. 15 shows a partially sectioned side view of another exemplary embodiment of an instrument configured in one-piece in accordance with the invention;

FIG. 16 shows a perspective view of the instrument according to FIG. 15;

FIG. 17 shows an enlarged view of a tip of the instrument according to FIGS. 15 and 16;

FIG. 18 shows a top view on another exemplary embodiment of an instrument in multi-piece configuration in accordance with the invention;

FIG. 19 shows a partially sectioned side view of the instrument according to FIG. 18;

FIG. 20 shows a perspective view of the instrument according to FIG. 18;

FIG. 21 shows an exploded view of the instrument according to FIG. 20;

FIG. 22 shows a perspective view of another one-piece exemplary embodiment of an instrument; and

FIG. 23 shows an enlarged view of a tip of the instrument according to FIG. 22.

### DETAILED DESCRIPTION

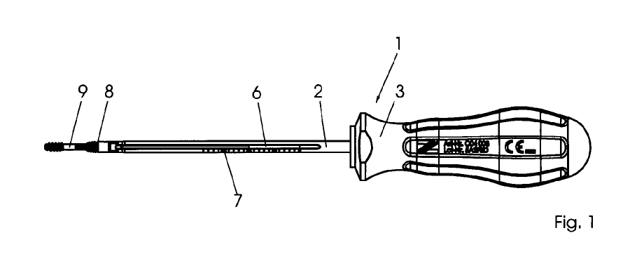

FIG. 1 shows a screwdriver with a cannulated shaft 2 and a grip 3, as a surgical instrument 1 in accordance with the invention.

The cannulated shaft 2 and the grip 3 can be either firmly connected or for example, interchangeably connected via a coupling.

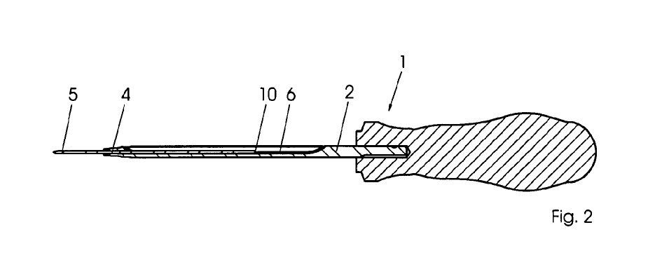

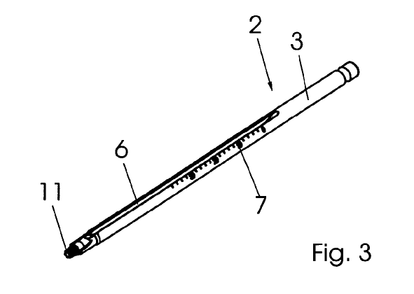

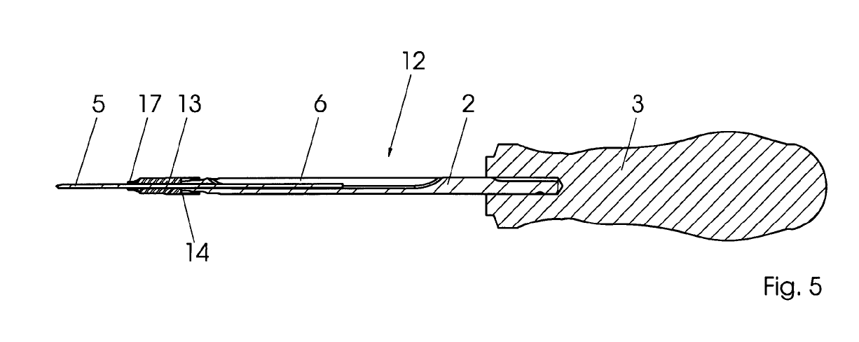

The cannulated shaft 2 is also represented in the FIGS. 2, 3, 4 and 5. As can be seen in the sectional representations of the FIGS. 2 and 5, the shaft 2 has a cannula 4. The cannula 4 is suitable for attaching a K-Wire 5 (so-called Kirschner Draht or Guide Wire) represented in the FIGS. 2 and 5. The cannulated shaft 2 has a slit 6, as represented in the FIGS. 1 to 5.

The slit 6 connects the cannula 4 with an outer surface of the shaft 2.

It should be noted for the manufacturing process the shaft 2 that in this option, initially a cannula is introduced and then the shaft is slit open up to the cannula. Furthermore, a method of manufacture can also be conceived, in which a thick shaft is slotted and subsequently closed again in a front section for guiding the K-Wire. The advantage of the second method manufacture is that the introduction of a long drill in the shaft can be dispensed with.

In addition, as represented in the FIGS. 1, 3 and 4, the shaft 2 has graduations 7. The graduations are disposed parallel to the slit 6.

In the exemplary embodiment of the surgical instrument 1 as an implant according to the FIG. 1, the shaft is connected to a bone screw 9 via a predetermined breaking point 8.

The function of the surgical instrument 1 according to the exemplary embodiment of the FIGS. 1, 2 and 3 is as follows:

In the shaft 2, a K-Wire 5 is inserted. As represented in FIG. 2, the length of the bone screw 9 and with this, the insertion depth of the shaft 2 over the position of an upper end 10 of the K-Wire 5 is displayed on the graduations 7.

The bone screw 9 is inserted by means of the surgical instrument 1 in a bone, until a certain torque is achieved and the bone screw 9 breaks off from the shaft 2 of the surgical instrument 1 at the predetermined breaking point 8. According to the functional principle of a Snap-Off-Screw, a carrier is freely laid in the screw head in the bone screw 9 after breaking off. If necessary, it can be gripped by a corresponding carrier 11 at the proximal end of the shaft 2 in the carrier in the screw head of the bone screw 9 and the bone screw can be screwed in still further.

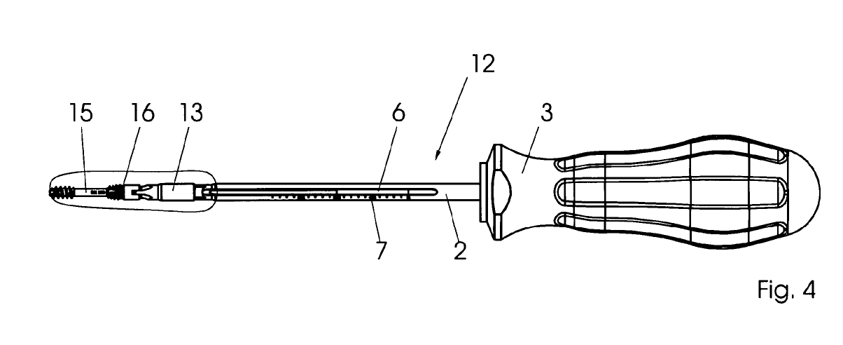

The FIGS. 4 and 5 show another exemplary embodiment of a surgical instrument 12. The surgical instrument is essentially configured similar to the surgical instrument 1 and likewise includes a cannulated shaft 2 and a grip 3.

In contrast to the exemplary embodiment of the surgical instrument 1, the surgical instrument 12 has an replaceable carrier 13. The replaceable carrier 13 can be connected to a proximal end 14 of the shaft 2 interchangeably as per the functionality of a bit. For this purpose, the proximal end 14 of the shaft 2 is preferably configured as a coupling element, which can be actively connected to the replaceable carrier 13 like a bit or a nut.

As represented in FIG. 4, the replaceable carrier 13 is connected to the bone screw 15 preferably via a predetermined breaking point 16.

The functionality is essentially similar to that of the previously described exemplary embodiment.

For the operator, there is the advantage with the surgical instrument 12 that he can have a usual grip 3 and a usual shaft 2, which he can combine in a simple manner by with a bone screw 15 which is connected to the carrier 13 via the predetermined breaking point 16. This offers the option of readily combining different bone screws or replaceable carriers 13 with the shaft 2 and the grip 3.

Similar to the exemplary embodiment of the surgical instrument 1, the bone screw 15 can be inserted further with a carrier 17 of the replaceable carrier 13 into the bone after the shearing at the predetermined breaking point 16. The carrier 17 of the replaceable carrier 13 is likewise initially freely laid on the predetermined breaking point 16 after the breaking off of the bone screw 15.

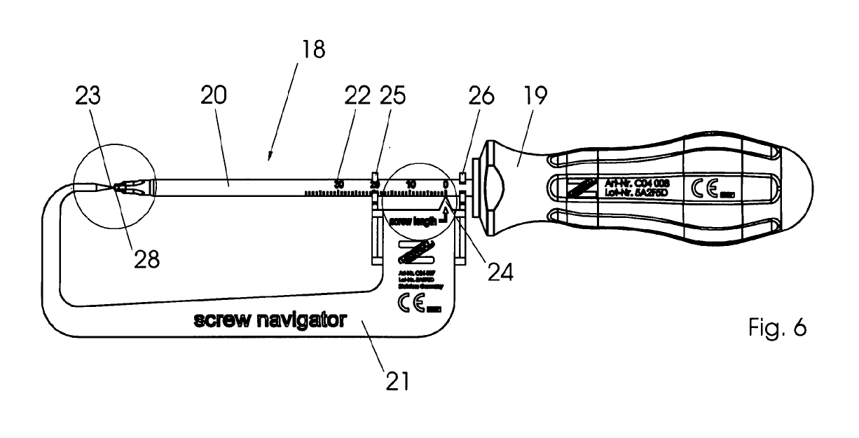

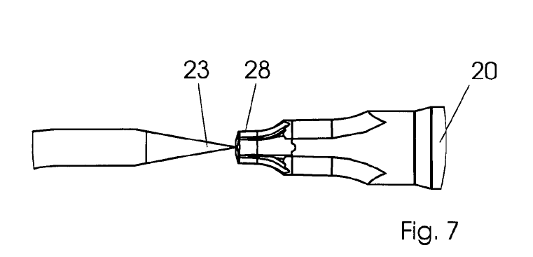

The FIGS. 6 to 12 disclose another exemplary embodiment of a surgical instrument 18. The surgical instrument includes a grip 19 and a shaft 20. In the represented exemplary embodiment, the shaft 20 does not have any cannula and any slit.

In further not represented exemplary embodiments, a surgical instrument similar to the surgical instrument 18, includes a shaft which has a cannula and a slit similar to the shaft 2 of the FIGS. 1 to 5.

A not represented exemplary embodiment of a surgical instrument in accordance with the invention includes a grip 19, a measuring caliper 21 and a shaft 2 according to the FIGS. 1-5.

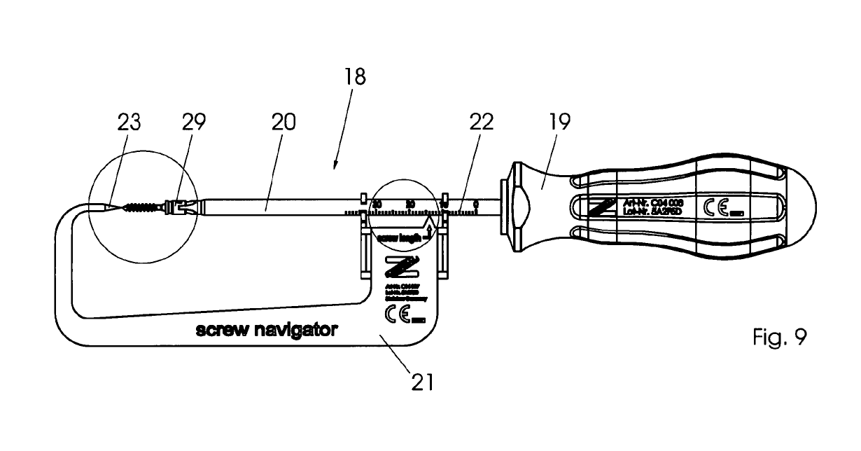

Furthermore, the surgical instrument 18 of the FIGS. 6 and 9 includes a measuring caliper 21. The shaft 20 has graduations 22 on its upper side.

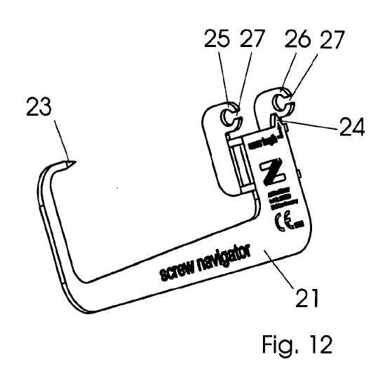

The measuring caliper 21 includes, as represented in FIG. 12, a test probe 23 and a display 24.

In addition, the measuring caliper 21 includes a first catch 25 and a second catch 26 on the shaft 20 or 2 as a moveable fixture of the measuring caliper 21. The catches 25 and 26 respectively have an opening 27. Preferably, the opening 27 respectively assumes only an angular range between 90° and 5°. The catches 25 and 26 can be suitably clipped over the shaft 20 or 2.

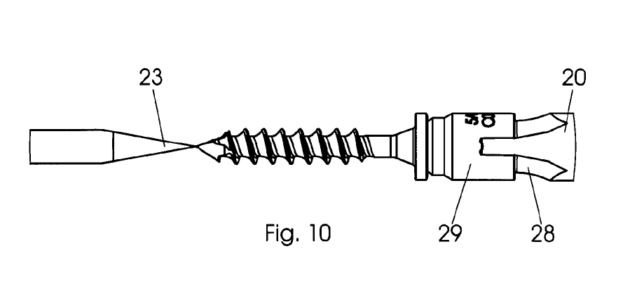

The shaft 20 has carrier 28, as represented in FIG. 6. The carrier is suitable for attaching a bone screw 29, as represented in the FIGS. 9 and 10.

In principle, the non-slit opened and non-cannulated shaft 20 can also be configured with a proximal end similar to the shaft 2 according to the FIGS. 1, 2, 3, 4 and 5. This means that a non-slit opened and non-cannulated shaft could likewise be connected via a predetermined breaking point with a bone screw, or a coupling, which is suitable for connecting with an replaceable carrier.

The functionality of the surgical instrument 18 according to the FIGS. 6 and 9 is as follows:

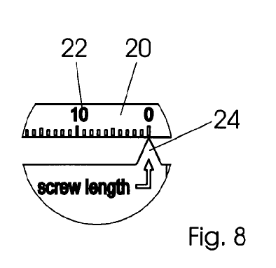

The measuring caliper 21 is mounted on the shaft 20 by means of the catches 25 and 26. Therefore, a tip 23 of the measuring apparatus 21 touches a front end of the carrier 28 of the shaft 20. A zero point is specified or determined on the graduations 22 of the shaft in the display 24 of the measuring apparatus 21, as represented in FIG. 8.

In further configurations here correct values can also be considered.

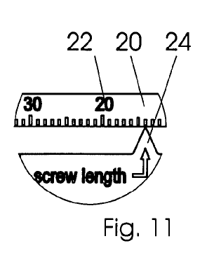

After attachment of the bone screw 9 to the carrier 28 of the shaft 20, as represented in the FIGS. 9 and 10, the tip 23 touches a tip of the bone screw 29. As a result, the measuring caliper 21 is shifted on the shaft 20 up to the display 24, as represented in FIG. 11, the length of the screw or an approximate value of the length of the screw is displayed on the graduations 22. The accuracy of the displayed screw-length value is always dependent on the depth of insertion of the carrier 28 on the screw-head of the bone screw 29.

In the exemplary embodiments not represented, the measuring caliper serves as a navigator for finding the correct screw position, as a fixture and/or counter-support during the screwing-in of the screw by hand or by a machine.

In typical exemplary embodiments not represented, the caliper has another tip, which facilitates the operator during pressing of the tip through skin and tissues on one side of the bone and by pressing of the screws of the tip on the other side of the bone to determine the length of the screw and its direction.

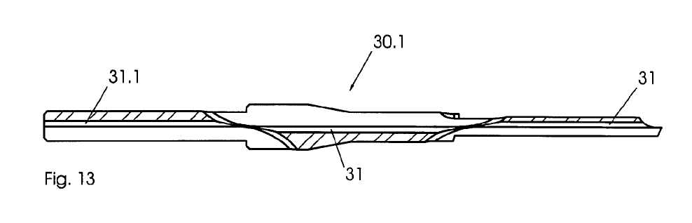

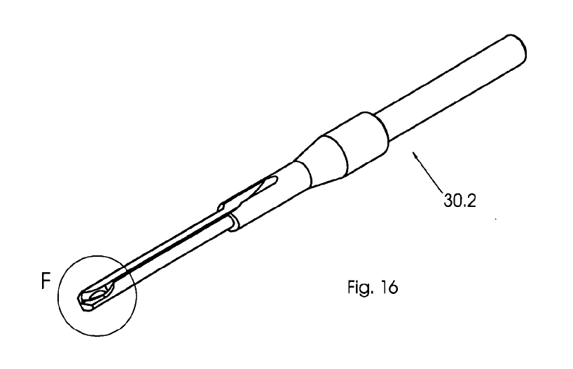

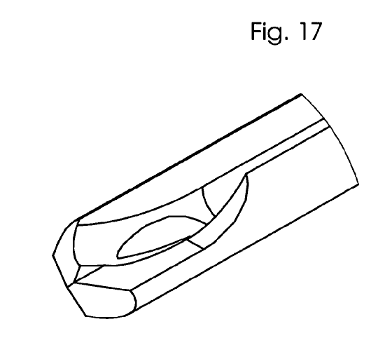

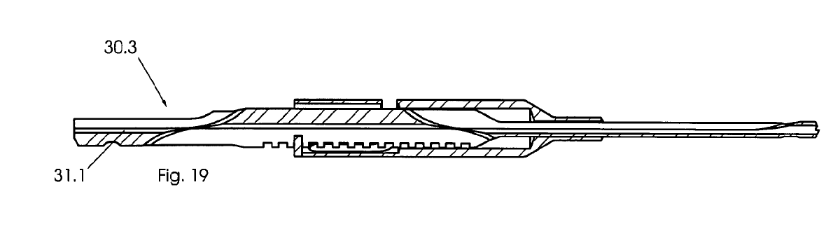

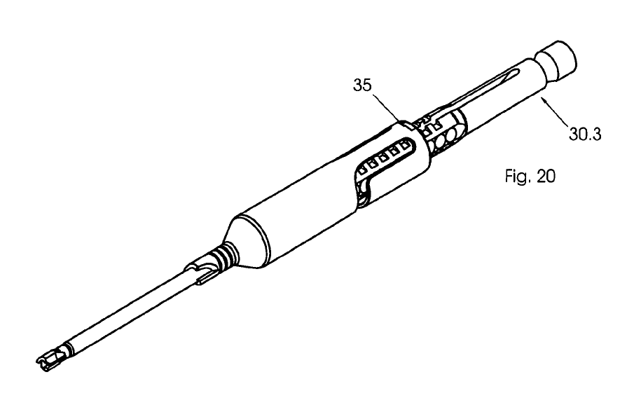

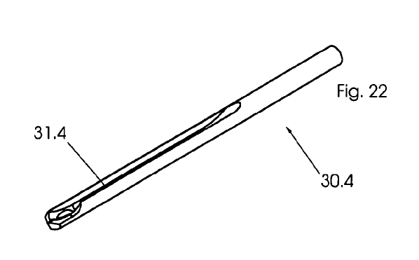

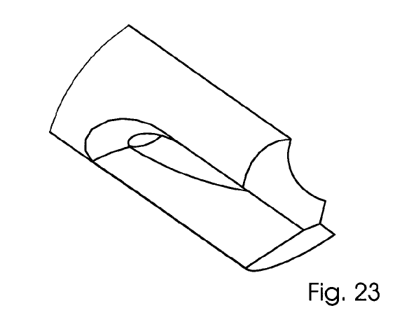

The instrument shown in the FIGS. 13 to 23 is preferably a screwing, drilling, countersinking, milling or impacting instrument. Presently, a drilling instrument 30.1 to 30.4 is represented, which is preferably configured cannulated (drilling instrument 30.1 and 30.3) and is partially slit open. The distances and/or at which angles the cannula and/or a slitting is provided, is different according to the application and instrument.

More preferably, at least one part should be cannulated and at least a part of one or more sides should be slit open, in order to ensure an optimum guidance of, e.g. a guide wire, not shown in more details.

The slits 31.1 to 31.4 can be made from several sides such that these connect preferably almost in the middle (axially) and thus form a cannula, e.g. for the guide wire. The cannula can also undertake further tasks, e.g. as a channel for insertion of a probe or as an anchor or other instruments.

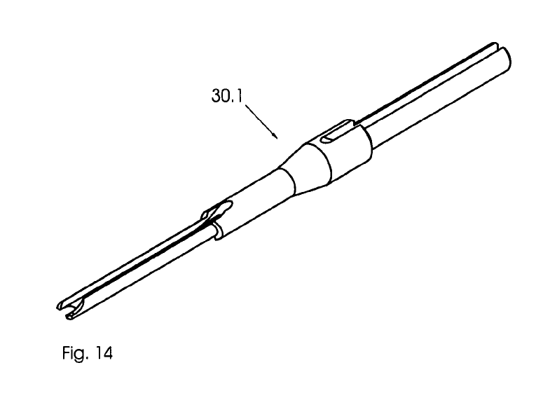

The FIGS. 14 to 16 and 22 and 23 show non-cannulated drilling instruments 30.2 and 30.4, in which spiral grooves on the outer sheath were dispensed with. Instead of this, these drilling instruments 30.2 and 30.4 are configured only partially slit opened, which is cheaper to manufacture, however ensures a comparative stability.

Preferably, in these drilling instruments 30.2 and 30.4, which are made from the slits 30.2 and 30.4 connected to at least one position or even pierce-through. Thus, e.g. during drilling, e.g. the material of a second drilling can likewise reach into the slit 30.2 and 30.4. Here, the advantage is that the material is collected in a slit-friendly manner and can be easily removed out of this, e.g. in order to be used at another position, or as sampling.

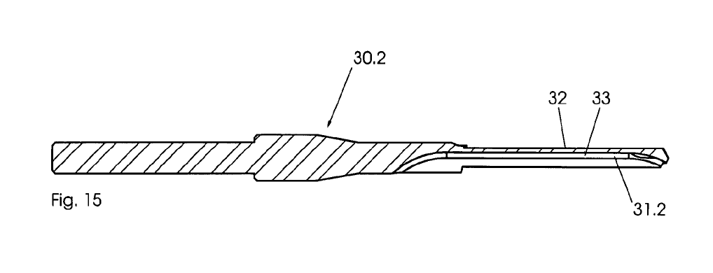

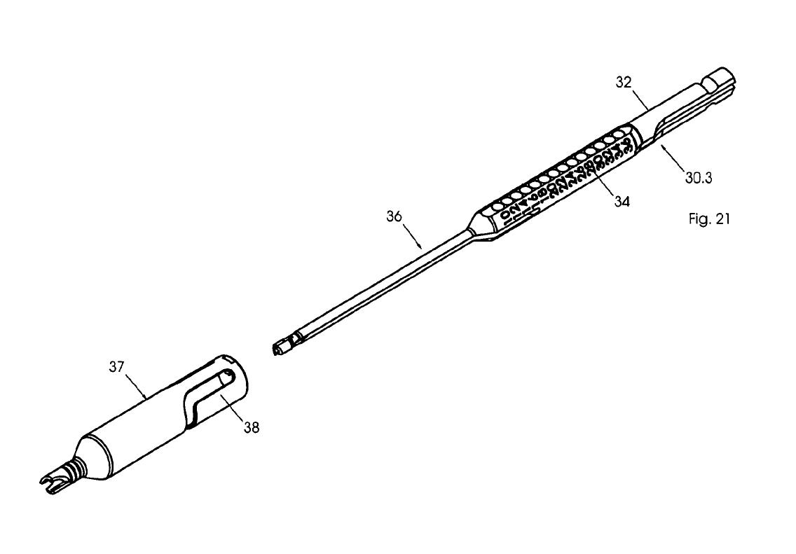

Furthermore, combinations with further configurations are possible. Thus, all instruments 30.1 to 30.4 can be generously provided with a markings 33 (see FIG. 15) and/or the graduations 34, for example, with colour markings (see e.g. FIG. 21) and/or labelling along the slots 31.1 to 31.4 on an outer sheath 32. Generally, these markings 33 and/or graduations 34 and/or labelling serve for determining the length of the or the depth of a guide wire introduced in a bone.

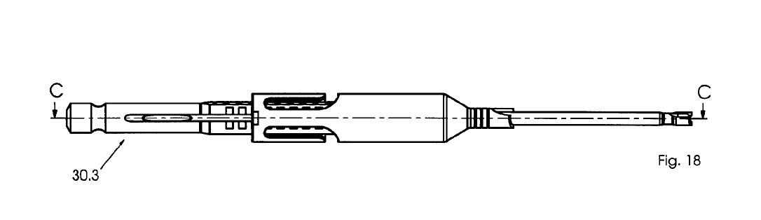

Furthermore, the instrument 30.3 can be, as shown in the FIGS. 18 to 21, equipped with a quick-latching system 35, in order to be able to easily adjust the screw-length. For this purpose, the drilling instrument 30.3 is provided with a base body 36 and a countersinking sleeve 37. The countersinking sleeve 37 has a resilient thrust piece 38, which causes latching of the thrust piece 38 with the base body 36, when the countersinking sleeve 37 is slid over the base body 36.

1-13. (canceled)

14. A surgical instrument (1, 12) for inserting an implant, the instrument comprises:
(a) a shaft (2), wherein the shaft is suitable for attaching the implant at the proximal end thereof, wherein the shaft (2) has a cannula; and
(b) a grip (3), which is connected to the shaft (2) at a distal end of the shaft (2),
wherein the shaft (2) is provided with a slit (6).

15. A surgical instrument (18) for inserting an implant, the instrument comprises:
(a) a shaft (20), wherein the shaft is suitable for attaching the implant at the proximal end thereof;
(b) a grip (19), which is connected to the shaft (20) at a distal end of the shaft (20); and
(c) a measuring caliper (21) suitable for determining a bore-depth and/or an implant-length.

16. A surgical instrument according to claim 14 wherein the shaft (2, 20) is provided with graduations (7, 22) suitable for determining the implant-length and/or the insertion-depth.

17. A surgical instrument according to claim 16, wherein the shaft (2, 20) is connected to the implant, at a proximal end of the shaft by a predetermined breaking point (8).

18. A surgical instrument according to claim 16, wherein the shaft (2) comprises a carrier (11) at a proximal end of the shaft, wherein the carrier is suitable to be brought in engagement with the implant.

19. A surgical instrument according to claim 16, wherein the shaft (2) comprises a coupling at a proximal end of the shaft, wherein the coupling is suitable for connecting with a carrier (13).

20. A surgical instrument according to claim 15, wherein the measuring caliper (21) comprises an end stop (23) to be applied on one of bone, skin or tissues of an implant end.

21. A surgical instrument according to claim 16, wherein the measuring caliper (21) comprises a marking (24), which indicates on the graduations (22) of the shaft (20), the measured length of an implant or insertion-depth.

22. A method of manufacture of a surgical instrument, comprising one of a screwdriver, a drill, a countersink, a milling-cutter, an instrument shaft, a guide sleeve, a guide tube with a cannula and an outer sheath, including the step of forming at least one slit (6) in the outer sheath, which forms at least in part the cannula.

23. A method according to claim 22, wherein the outer sheath has a length and including forming the slit at least partially over the entire length of the outer sheath.

24. A method according to claim 22, including inserting at least one drill at a distal or a proximal end of the surgical instrument.
